SEQUENCE LISTING (20 sequences)

SEQ ID NO: 1 - protein, 45 aa, unidentified
ARGTHRGLNLEUPROPROALATYRTHRASNSERPHETHRARGGLY

SEQ ID NO: 2 - protein, 45 aa, unidentified
LEUGLYVALTYRTYRHISLYSASNASNLYSSERTRPMETGLUSER

SEQ ID NO: 3 - protein, 45 aa, unidentified
GLYALAALAALATYRTYRVALGLYTYRLEUGLNPROARGTHRPHE

SEQ ID NO: 4 - protein, 45 aa, unidentified
CYSALALEUASPPROLEUSERGLUTHRLYSCYSTHRLEULYSSER

SEQ ID NO: 5 - protein, 45 aa, unidentified
ALAPROGLYGLNTHRGLYLYSILEALAASPTYRASNTYRLYSLEU

SEQ ID NO: 6 - protein, 45 aa, unidentified
LEULYSPROPHEGLUARGASPILESERTHRGLUILETYRGLNALA

SEQ ID NO: 7 - protein, 45 aa, unidentified
PROPHEGLNGLNPHEGLYARGASPILEALAASPTHRTHRASPALA

SEQ ID NO: 8 - protein, 45 aa, unidentified
ASPTHRTHRASPALAVALARGASPPROGLNTHRLEUGLUILELEU

SEQ ID NO: 9 - protein, 45 aa, unidentified
THRLEUGLUILELEUASPILETHRPROCYSSERPHEGLYGLYVAL

SEQ ID NO: 10 - protein, 45 aa, unidentified
GLUCYSASPILEPROILEGLYALAGLYILECYSALASERTYRGLN

SEQ ID NO: 11 - protein, 45 aa, unidentified
TYRILECYSGLYASPSERTHRGLUCYSSERASNLEULEULEUGLN

SEQ ID NO: 12 - protein, 45 aa, unidentified
LYSPROSERLYSARGSERPHEILEGLUASPLEULEUPHEASNLYS

SEQ ID NO: 13 - protein, 45 aa, unidentified
LEULEUPHEASNLYSVALTHRLEUALAASPALAGLYPHEILELYS

SEQ ID NO: 14 - protein, 45 aa, unidentified
SERPROASPVALASPLEUGLYASPILESERGLYILEASNALASER

SEQ ID NO: 15 - protein, 45 aa, unidentified
GLNGLULEUGLYLYSTYRGLUGLNTYRILELYSTRPPROTRPTYR

SEQ ID NO: 16 - protein, 45 aa, unidentified
SERASNGLULYSGLNGLUILELEUGLYTHRVALSERTRPASNLEU

SEQ ID NO: 17 - protein, 45 aa, unidentified
HISHISALAASNGLUTYRARGLEUTYRLEUASPALATYRASNMET

SEQ ID NO: 18 - protein, 48 aa, unidentified
METASNSERARGASNTYRILEALAGLNVALASPVALVALASNPHEASN

SEQ ID NO: 19 - protein, 45 aa, unidentified
METLYSILEILELEUPHELEUALALEUILETHRLEUALATHRCYS

SEQ ID NO: 20 - protein, 33 aa, unidentified
THRLEUCYSPHETHRLEULYSARGLYSILEGLU

(19) United States
(12) Patent Application Publication
(10) Pub. No.: US 2024/0279286 A1
(43) Pub. Date: Aug. 22, 2024

(54) CORE AMINO ACID SEQUENCE GROUP CAPABLE OF TARGET RECOGNIZING ANTI-NOVEL CORONAVIRUS NEUTRALIZING ANTIBODIES N-IGY-PABS AND USE THEREOF

(71) Applicant: SINO-SWED TONGKANG BIO-TECH (SHENZHEN) LIMITED, Shenzhen (CN)

(72) Inventors: Ailian HEI, Shenzhen (CN); Jin LI, Shenzhen (CN); Ellen HE, Shenzhen (CN); Sven Isac SKOG, Shenzhen (CN); Ji ZHOU, Shenzhen (CN)

(21) Appl. No.: 18/636,457

(22) Filed: Apr. 16, 2024

Related U.S. Application Data
(63) Continuation of application No. PCT/CN2022/126859, filed on Oct. 22, 2022.

(30) Foreign Application Priority Data
Oct. 22, 2021 (CN) 202111232491.9

(51) Int. Cl. C07K 14/005; G01N 33/569; G01N 33/68

(52) U.S. Cl. CPC C07K 14/005; G01N 33/56983; G01N 33/6854; C12N 2770/20022

(57) ABSTRACT

Specifically disclosed are a core amino acid sequence group capable of target recognizing anti-novel coronavirus neutralizing antibodies N-IgY-pAbs and a use thereof. The core amino acid sequence group capable of target recognizing anti-novel coronavirus neutralizing antibodies N-IgY-pAbs includes 15 types of the amino acid sequences positioned in an S-ECD region and 5 types of the amino acid sequences positioned in a non-structural protein (NSP) region, and can be applied to a detection of the novel coronavirus, a design of therapeutic targets and a design of vaccine targets.

### CROSS-REFERENCE TO RELATED APPLICATIONS

The present application is a continuation of PCT application No. PCT/CN2022/126859, filed on Oct. 22, 2022, which claims the priority and benefit to Chinese patent application No. 202111232491.9, filed on Oct. 22, 2021. The entireties of PCT application No. PCT/CN2022/126859 and Chinese patent application No. 202111232491.9 are hereby incorporated by reference herein and made a part of this specification.

### REFERENCE TO AN ELECTRONIC SEQUENCE LISTING

The contents of the electronic sequence listing (SequenceListing.xml; Size: 19,028 bytes; and Date of Creation: Apr. 16, 2024) is herein incorporated by reference.

### TECHNICAL FIELD

The present application relates to a field of bio-medicine, and in particular, to a core amino acid sequence group capable of target recognizing anti-novel coronavirus neutralizing antibodies N-IgY-pAbs (neutralizing IgY polyclonal antibodies) and a use thereof.

### BACKGROUND ART

Epidemic of novel coronavirus pneumonia (simply named as COVID-19) is the most concerned emerging infectious diseases event in the world today. Since the epidemic outbreak around the world, it has not been effectively and completely contained. The novel coronavirus (referred to as COVID-19) has infected a large number of people, is highly transmissible, and has a high case fatality rate. As so far, a great progress has been made in the development of COVID-19 vaccines and treatment means. The vaccine has been popularized and applied to the market, however, there is still no specific therapeutic drug for the novel coronavirus. Therefore, it is still urgent to explore new methods, new means and new instruments for the prevention, diagnosis and treatment of the epidemic of novel coronavirus pneumonia.

Based on basic immunological mechanisms of virus infection and antibody production, use of novel coronavirus-specific antibody can neutralize virus, thereby preventing the virus from adhering to and invading host cells. The novel coronavirus infects organism by first binding to receptor angiotensin converting enzyme 2(ACE2) of host cell through receptor binding domain (RBD) on spike protein (S protein), which mediates the virus entry into the host cell. Therefore, in an aspect of immunogen selection, an S protein extracellular domain (S-ECD) of the novel coronavirus is adopted as an ideal and effective immunogen, which can effectively induce a production of neutralizing antibodies.

In terms of antibody types, IgY antibody of poultry or bird has advantages. IgY passive immunotherapy strategy has been used to prevent and treat pathogen infections in humans and animals. Among them, the S protein of virus is a preferable target protein for respiratory coronavirus antibody drugs. In the development of therapeutic drugs for respiratory virulent viral infectious diseases that occurred in the past, such as middle east middle east respiratory syndrome coronavirus (MARS-CoV), some scholars used recombinant MERS-CoV S subunit protein to prepare IgY polyclonal antibody, and found that IgY antibodies can effectively neutralize an infection effect of MERS-CoV through a neutralization test in vitro and an animal model test in vivo. There are also some studies using IgY polyclonal antibody prepared from nucleocapsid protein (N protein), which also shows a strong high affinity with the N protein. However, it is still unknown that which specific target sites in novel coronavirus proteome are recognized by the IgY polyclonal antibody.

The anti-novel coronavirus antibodies can generally be divided into two categories, namely Neutralizing Antibodies (NAbs) and non-neutralizing virus binding antibodies (BAbs, Binding Antibodies). The S protein of the novel coronavirus is a key target of the NAbs, since the novel coronavirus invades the host cells through the interaction of its S protein thereof and the ACE2 protein on the surface of the host cell. While the BAbs can bind to all protein components of the novel coronavirus, including S, N, E and M protein. NAbs are one of the most important criteria for predicting the success of a novel corona vaccine.

At the current situation of preventing and controlling the novel coronavirus, although the novel coronavirus vaccine has been developed and marketed for coping with a pandemic of the novel coronavirus pneumonia, there is no specific therapeutic drugs for the novel coronavirus at present. During the continuous development of novel coronavirus vaccines and drugs, a serious challenge currently faced is frequent mutations of the novel coronavirus, which may lead to an immune escape or an enhanced adaptability of the virus. New mutation strain is easily to cause a recurrence of the epidemic, weaken a protective effect of the vaccine, thereby worsening the global spread of the epidemic. Therefore, regarding to the mutation of the virus, mutation hotspot regions should be avoided, and conserved regions and sequences should be selected in designing detection and treatment targets for the novel coronavirus.

From December 2019 to December 2020, the novel coronavirus mutated at different speeds. Researchers analyzed mutation rates of 27 different proteins at different periods, found that the mutation rates of different protein of the novel coronavirus were totally different, and observed that the S protein and N protein, such as D614G (S protein), P323L (NSP12) and R203K/G204R (N protein), had the highest mutation variability in the vaccine and treatment of the novel coronavirus. Recent mutations have identified additional sites, such as A222V (S protein), L18F(S protein) and A220V (N protein) (Vilar S, Isom D G. One Year of SARS-CoV-2:How Much Has the Virus Changed?. Biology,2021,10:91). For recent Delta variant of the novel coronavirus, the mutation of which has at least 13 sites, mainly positioned in the S protein, such as D614G, T478K, L452R, P681R and E484Q (S protein).

In the development and research of the novel coronavirus vaccines and therapeutic drugs, the difficulty of selecting target sequences on the S protein and the N proteins, especially for the S proteins, is increased because main targets and research focus are on the S protein and N protein, which have the highest mutation variability.

Therefore, the inventor believes that in an evaluation of specific binding target sequences of the antibody, whether a mutation region exists and whether a mutation point is included is an important index for evaluating an efficiency. Faced with the current unfavorable situation of high-frequency mutations of the novel coronavirus, finding of a conserved amino acid sequence bound with a specific antibody, especially a neutralizing antibody on the S protein has important significance and application value.

### SUMMARY

In order to effectively cope with the current unfavorable situation of high-frequency mutations of the novel coronavirus, the present application provides a core amino acid sequence group capable of target recognizing anti-novel coronavirus neutralizing antibodies N-IgY-pAbs and a use thereof.

In a first aspect, the present application provides a core amino acid sequence group capable of target recognizing anti-novel coronavirus neutralizing antibodies N-IgY-pAbs, adopting the following technical solution.

A core amino acid sequence group capable of target recognizing anti-novel coronavirus neutralizing antibodies N-IgY-pAbs includes the following amino acid sequences:

SNTD:
²¹RTQLPPAYTNSFTRG³⁵,
¹⁴¹LGVYYHKNNKSWMES¹⁵⁵,
²⁶¹GAAAYYVGYLQPRTF²⁷⁵
and
²⁹¹CALDPLSETKCTLKS³⁰⁵;
S-RBD:
⁴¹¹ APGQTGKIADYNYKL⁴²⁵
and
⁴⁶¹LKPFERDISTEIYQA⁴⁷⁵;
S-CTD1:
⁵⁶¹PFQQFGRDIADTTDA⁵⁷⁵,
⁵⁷¹DTTDAVRDPQTLEIL ⁵⁸⁵
and
⁵⁸¹TLEILDITPCSFGGV⁵⁹⁵;
S-CTD2:
⁶⁶¹ECDIPIGAGICASYQ⁶⁷⁵;
S1/S2 border region:
⁷⁴¹YICGDSTECSNLLLQ⁷⁵⁵,
⁸¹¹KPSKRSFIEDLLFNK⁸²⁵
and
⁸²¹LLFNKVTLADAGFIK ⁸³⁵;
S-HR2:
¹¹⁶¹SPDVDLGDISGINAS¹¹⁷⁵;
and
S-HR2-TM:
¹²⁰¹QELGKYEQYIKWPWY¹²¹⁵,

positioned in an S-ECD region and numbered according to a protein sequence; and

ORF1ab:
¹³⁶¹SNEKQEILGTVSWNL¹³⁷⁵;
ORF1ab:
⁶⁴¹¹HHANEYRLYLDAYNM⁶⁴²⁵;
ORF10:
²¹MNSRNYIAQVDVVNFNLT³⁸;
and
ORF7a:
¹MKIILFLALITLAT¹⁵
and
¹¹¹TLCFTLKRKTE¹²¹,

positioned in a non-structure protein region and numbered according to a protein sequence.

In some embodiments, the core amino acid sequence group capable of target recognizing anti-novel coronavirus neutralizing antibodies N-IgY-pAbs consists of the following amino sequences:

SNTD:
²¹RTQLPPAYTNSFTRG³⁵,
¹⁴¹LGVYYHKNNKSWMES¹⁵⁵,
²⁶¹GAAAYYVGYLQPRTF²⁷⁵
and
²⁹¹CALDPLSETKCTLKS³⁰⁵;
S-RBD:
⁴¹¹APGQTGKIADYNYKL⁴²⁵
and
⁴⁶¹LKPFERDISTEIYQA⁴⁷⁵;
S-CTD1:
⁵⁶¹PFQQFGRDIADTTDA⁵⁷⁵,
⁵⁷¹DTTDAVRDPQTLEIL⁵⁸⁵
and
⁵⁸¹TLEILDITPCSFGGV⁵⁹⁵;
S-CTD2:
⁶⁶¹ECDIPIGAGICASYQ⁶⁷⁵;
S-HR2-TM:
¹²⁰¹QELGKYEQYIKWPWY¹²¹⁵
S1/S2 border region:
⁷⁴¹YICGDSTECSNLLLQ⁷⁵⁵
⁸¹¹KPSKRSFIEDLLFNK⁸²⁵
and
⁸²¹LLFNKVTLADAGFIK⁸³⁵;
S-HR2:
¹¹⁶¹SPDVDLGDISGINAS¹¹⁷⁵;
and
S-HR2-TM:
¹²⁰¹QELGKYEQYIKWPWY¹²¹⁵,

positioned in the S-ECD region and numbered according to the protein sequence; and

ORF1ab:
¹³⁶¹SNEKQEILGTVSWNL¹³⁷⁵;
ORF1ab:
⁶⁴¹¹HHANEYRLYLDAYNM⁶⁴²⁵;
ORF10:
²¹MNSRNYIAQVDVVNFNLT³⁸;
and
ORF7a:
¹MKIILFLALITLAT¹⁵
and
¹¹¹TLCFTLKRKTE¹²¹,

positioned in the non-structure protein region and numbered according to the protein sequence.

Grading mutation frequencies (MRs) of residues in the novel coronavirus from an extremely low mutation frequency to a low mutation frequency by a three-dimensional protein structure analysis into 3 grades, MRs=0.01-0.025, MRs=0.025-0.05 and MRs=0.05-0.10, respectively; and when MRs>0.20, the mutation frequency is high. In the above 20 types of target amino acid sequences effectively recognized by neutralizing antibodies, 15 types of which are identified to be positioned in the S-ECD region, and 5 types of which are positioned in the non-structural protein (NSP) region. Among them, P272 is found to belong to residues with the low mutation frequency only in one S protein aa261-275, and the other 19 types of the core amino acid sequences all belong to conserved amino acid sequences, do not contain currently discovered viral mutation sites, have high conservative, and can be effectively used for coping with the current unfavorable situation of high-frequency mutations of the novel coronavirus.

In some embodiments, the amino acid sequence is an adjusted or modified amino acid sequence, a material used for modification includes any one or more of selected from a group consisting of nanomaterial, fluorescent material, enzyme, biotin, and specific protein.

By adopting the above technical solution, one or more of the above amino acid sequences are used as a core to perform corresponding adjustment or modification, and the material used for modification includes but is not limited to the nanomaterial, the fluorescent material, the enzyme, the biotin and the specific protein, which is beneficial to apply the adjusted or modified amino acid sequences to a detection of the novel coronavirus, a design of immune antigens of novel coronavirus vaccines, an evaluation of the novel coronavirus vaccines and so on.

In a second aspect, the present application provides a use of the core amino acid sequence group capable of target recognizing anti-novel coronavirus neutralizing antibodies N-IgY-pAbs, adopting the following technical solution.

Use of the core amino acid sequence group capable of target recognizing anti-novel coronavirus neutralizing antibodies N-IgY-pAbs, using one or more of the amino acid sequences as a core, is used for a detection of the novel coronavirus, or used in preparing a reagent or a kit for detecting the novel coronavirus.

By adopting the above technical solution, the above amino acid sequences have high specificity and high affinity characteristics, and can be effectively applied to quantitative and/or qualitative detection of the novel coronavirus by using one or more of the amino acid sequences as the core.

In some embodiments, the detection includes but is not limited to ELISA detection, chemiluminescence immunoassay detection and immunofluorescence method detection.

By adopting the above technical solution, the amino acid sequences can be used for various detection methods such as the ELISA detection, the chemiluminescence immunoassay detection and the immunofluorescence method detection, with a wide range of applications and strong applicability.

Use of the core amino acid sequence group capable of target recognizing anti-novel coronavirus neutralizing antibodies N-IgY-pAbs, using one or more of the amino acid sequences as a core, is used in a design of therapeutic targets for the novel coronavirus.

By adopting the above technical solution, the above amino acid sequences have high specificity and high affinity characteristics, using one or more of the amino acid sequences as the core, are applied to the design of therapeutic targets for the novel coronavirus, and a therapeutic formulation for the novel coronavirus can be designed.

In some embodiments, the design of therapeutic targets includes but is not limited to a design of targets of therapeutic antibodies and a design of targets of non-antibody therapeutic drugs.

By adopting the above technical solution, the amino acid sequences can be used to the design of targets of therapeutic antibodies and the design of targets of non-antibody therapeutic drugs, with a wide range of applications and strong applicability.

Use of the core amino acid sequence group capable of target recognizing anti-novel coronavirus neutralizing antibodies N-IgY-pAbs, using one or more of the amino acid sequence as a core, is used to a design of vaccine targets for the novel coronavirus.

By adopting the above technical solution, the above amino acid sequences have high specificity and high affinity characteristics, using one or more of the amino acid sequences as the core, and can be applied to the design of vaccine targets for the novel coronavirus, and can be effectively used for coping with the current unfavorable situation of high-frequency mutations of the novel coronavirus.

In some embodiments, the design of vaccine targets includes but is not limited to a design of vaccine immune antigens and an evaluation of vaccine performance.

By adopting the above technical solution, the amino acid sequences can be applied to the design of vaccine immune antigens and the evaluation of vaccine performance, with a wide range of applications and strong applicability.

In summary, the present application has the following beneficial effects.
1. Among 20 types of the amino acid sequences provided in the present application, only one amino acid sequence contains an extremely low mutation frequency site in the S protein (S protein P272), the other 19 types of the amino acid sequences are all high-conservative amino acid sequences, do not contain currently discovered viral mutation sites, have high conservative, and can be effectively used for coping with the current unfavorable situation of high-frequency mutations of the novel coronavirus.
2. In the present application, 20 types of high specificity and high affinity amino acid sequences can be used to effectively design detection formulations for the novel coronavirus, as well as therapeutic formulations and vaccine formulations for the novel coronavirus.

### BRIEF DESCRIPTION OF THE DRAWINGS

FIG. 1 is a detection diagram of a novel coronavirus proteome chip of Examples in the present application, which detects an immune response of anti-novel coronavirus neutralizing antibodies N-IgY-pAbs recognizing different polypeptide binding sites, N-IgY-pAbs (375 ng/ml).

FIG. 2 is a detection diagram of a novel coronavirus proteome chip of Examples in the present application, which detects an immune response of non-immunized hen serum recognizing different polypeptide binding sites, non-immunized hen serum (1:2000).

FIG. 3 is a detection diagram of a novel coronavirus proteome chip of Examples in the present application, which is obtained an immune response distribution chart according to Z score >0.05, in which the Z score >3.0 is considered to be a significantly strong signal, and the Z score >5.0 is considered to be a significant peak signal.

FIG. 4 is a schematic diagram of an S-ECD protein sequence (1273 amino acid residues). The S-ECD in figure shows: two main domains of S1 (head) and S2 (stem). Different structural domains are marked with different colors, and a primary structure sequence distribution is named as following: SP (starting paragraph), NTD (N-terminal domain); RBD (receptor binding domain) including RBM (receptor binding motif), CTD1 (C-terminal structural domain 1), CTD2 (C-terminal structural domain 2), S1/S2 border (S1/S2 junction), S2′ (S2′cleavage site), FL (fusion loop), FPPR (fusion peptide proximal region), HR1 (heptapeptide repeat 1), CE (center helix region), CD (connector domain), HR2 (hetapeptide repeat 2), TM (transmembrane domain) and CT (C terminal).

FIG. 5 is amino acid sequences with the Z score >0.05 in other structural domains expect the S-ECD, in which a non-structural protein domain has 5 types of the amino acid sequences, and the Z score >3.0 is considered to be a significantly strong signal.

### DETAILED DESCRIPTION

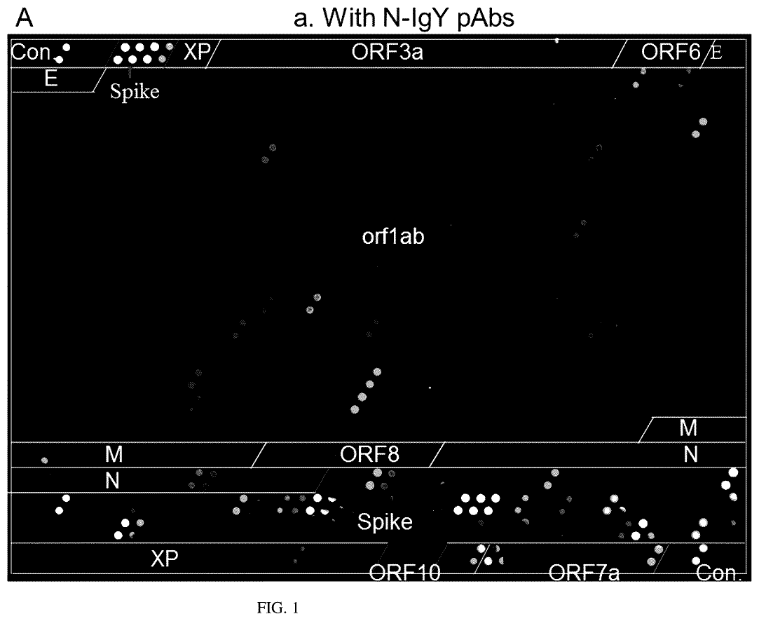

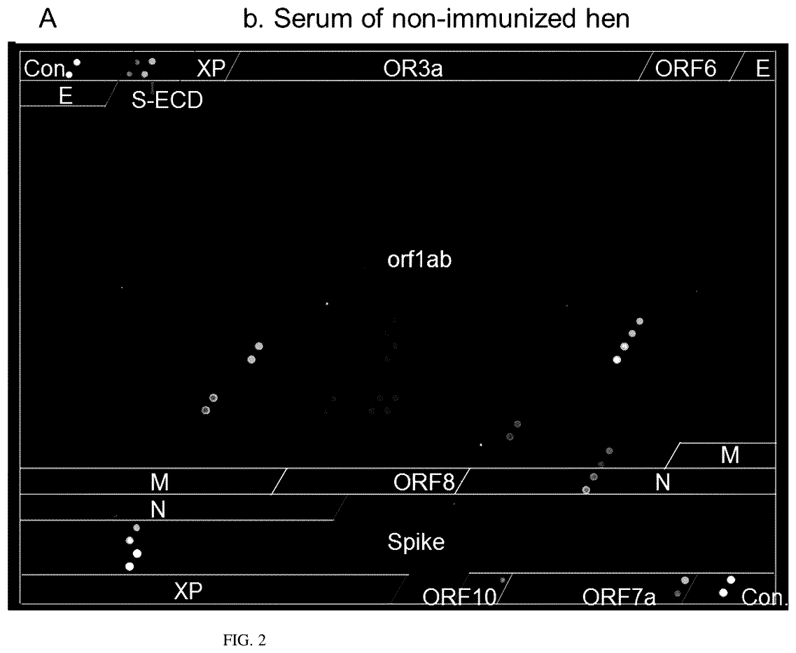

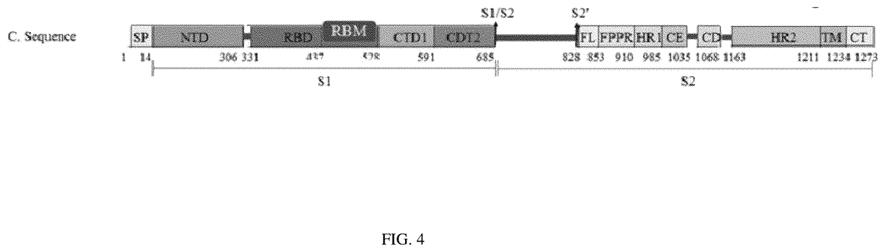

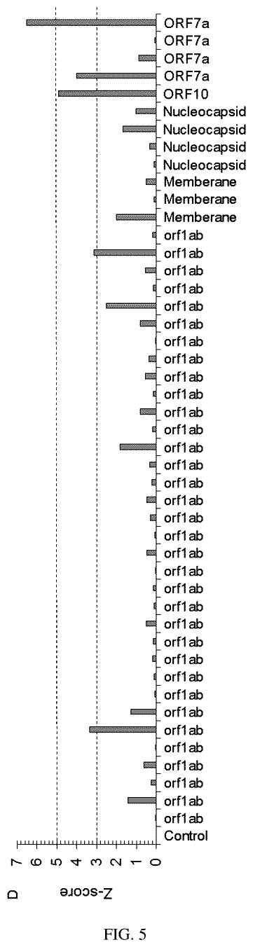

The present application is further described in detail below in combination with FIGS. 1-5 and Examples. It should be noted that, if specific conditions are not specified in the following Examples, the conditions should be carried out according to conventional conditions or conditions recommended by the manufacturer. Unless otherwise specified, raw materials used in the following Examples can all be obtained from ordinary commercial sources.

### EXAMPLE

### Example 1

### 1. Preparation of the Anti-Novel Coronavirus Neutralizing Antibodies N-IgY-pAbs.

The preparation method of the anti-novel coronavirus neutralizing antibodies N-IgY-pAbs proposed in the Chinese invention patent with announcement number CN112094341B was adopted. Hens were immunized by using a spike protein extracellular domain (S-ECD) of the novel coronavirus, yolk antibodies were extracted, and the novel coronavirus neutralizing antibodies N-IgY-pAbs were screened and prepared.

### 2. Preparation of a Novel Coronavirus Proteome Chip.

All biotin-labeled polypeptides were completed by Shanghai Qiangyao Biotechnology Co., Ltd. and Guoping Pharmaceutical Company. All novel coronavirus E, N and S proteins were purchased from Beijing Yiqiao Shenzhou Technology Co., Ltd. Polypeptides and proteins (Table 1) parallelly printed on a surface of a 3D modified glass slide (provided by Biobio), and spotted by using an Arrayjet biochip spotter. A polypeptide chip was stored at −20° C. until ready for used.

TABLE 1
Sequence regions and names of controls, polypeptides
and proteins spotted on a slide matrix

| The sequence | |
|---|---|
| regions spotted | |
| on the slide matrix | Name |
| 1 positive control | Human poliovirus polypeptide, 200 μg/mL |
| 2 negative control | Human hemagglutinin polypeptide, 200 μg/mL |
| 3 positive control | Mixture of IgG and IgM, 200 μg/mL |
| 4 negative control | Streptavidin, 200 μg/mL |
| 5 negative control | BSA, 200 μg/mL |
| 6-10 protein | N, S-ECD, S1, S2, RBD, as positive quality |
| | control |
| 11-13 polypeptide | XP27-29 (proteins unrelated to the new |
| region | coronavirus), as negative quality control |
| 14-40 polypeptide | ORF3a: 1-27# |
| region | |
| 41-46 polypeptide | ORF6: 1-6# |
| region | |
| 47-53 polypeptide | Envelope (E): 1-7# |
| region | |
| 54-762 polypeptide | Orflab: 1-709# |
| region | |
| 763-784 polypeptide | Membrane (M): 1-22# |
| region | |
| 785-796 polypeptide | ORF8: 1-12# |
| region | |
| 797-837 polypeptide | Nucleocapsid (N): 1-41# |
| region | |
| 838-960 polypeptide | Spike(S): 1-123# |
| region | |
| 961-984 polypeptide | XP: 2&3#, 4&5#, 6-26# (proteins unrelated to the |
| region | novel coronavirus) |
| 985-988 polypeptide | Spike(S): 124-127# |
| region | |
| 989-991 polypeptide | ORF10: 1-3# |
| region | |
| 992-1003 polypeptide | ORF7a: 1-12# |
| region | |
| 1004-1008 control | BSA, streptavidin, Mixture of IgG and IgM, |
| | Human hemagglutinin polypeptide, Human |
| | poliovirus polypeptide |

3. Whole Proteome Scanning of Binding Targets for Anti-Novel Coronavirus N-IgY pAbs.
(1) Hybridization reaction of the N-IgY pAbs: The proteome chips were placed in culture dishes, and blocked with PBST containing 5%(w/v) milk and 0.2%(v/v) Tween-20 at room temperature for 10 minutes. After washing, microarrays 1 and 2 were incubated with the N-IgY-pAbs, respectively (concentrations of the N-IgY-pAbs were 375 ng/ml and 186 ng/mL, respectively). At the same time, pre-immunized hen serum was added into microarray 3 (serum dilution 2000×) and microarray 4 (serum dilution 4000×), both incubated for 30 minutes. Subsequently, after washing three times, the chip was incubated with a goat-anti-chicken secondary antibody labeled with Alexa Fluor 555 (Abcam, USA) at room temperature for 20 minutes. Then the chip was dried by a vacuum pump.
(2) Data analysis: The proteome chip was scanned by a GenePix 4300A chip scanner (Molecular Devices, USA), and results of the scanning and analysis were shown in FIGS. 1-5.

A median fluorescence signal intensity of each spot was extracted by GenePix Pro7 software (Molecular Devices, USA). The raw fluorescence signal intensity was that a median signal intensity of each spot minus a median background intensity of each spot, and an average value of duplicate wells was calculated. The resulting signal was normalized by using the Z-score. Higher Z-score indicates stronger reaction signal, and more specific recognition and stronger affinity of the anti-novel coronavirus neutralizing antibodies N-IgY-pAbs.

The Z score >3.0 was considered to be a significantly strong signal, and the Z score >5.0 was considered to be a significant peak signal. The result showed that, four negative controls in the control group had no signal, and only the mixture of IgG and IgM in two positive control groups showed stronger positive, but the human poliovirus polypeptide had no signal, indicating that the anti-novel coronavirus neutralizing antibodies N-IgY-pAbs do not recognize the human poliovirus polypeptide. Since the human poliovirus polypeptide was unrelated to the novel coronavirus, no signal was displayed. The anti-novel coronavirus neutralizing antibodies N-IgY-pAbs could significantly recognize the positive quality controls of proteins of S1+S2, S1 and S2, reaching the significant peak signal (Z score) ≥5.0, verifying a high sensitivity and high specificity of the anti-novel coronavirus neutralizing antibodies N-IgY-pAbs.

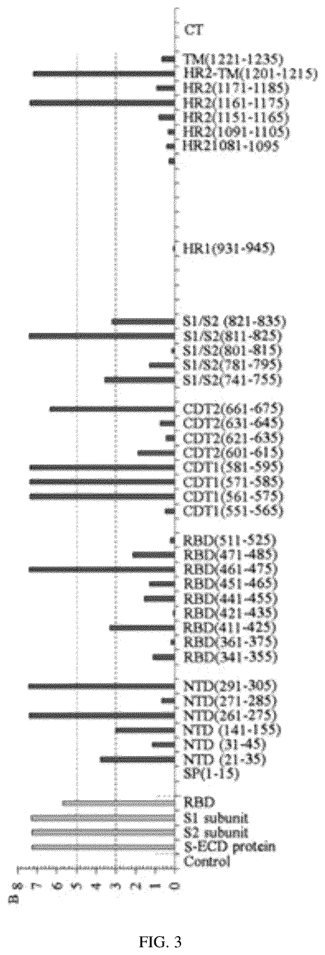

Referring to FIG. 3, there were 20 potent targets identified as the significantly strong signal (the Z score ≥3.0), and there were 11 highly effective target peptides identified as the significant peak signal (the Z score ≥5.0).

Sorted by the Z score, the reaction signals of 20 types of the amino acid sequences with the Z scores >3.0 on the proteome chip were shown in Table 2. Among them, only one amino acid sequence contained a low mutation frequency site in the S protein (MRs<0.025, and S-NTD protein P272), the other 19 types of the amino acid sequences were all high-conservative sequences and do not contain currently discovered viral mutation sites, and can be used for designing, developing and researching the anti-novel coronavirus antibodies with high mutation frequency.

TABLE 2
Reaction signal on the proteome chip and core amino acid sequences
information capable of target recognizing N-IgY-pAbs

| | Z Score | | | | |
|---|---|---|---|---|---|
| | Novel | Natural | | | |
| | coronavirus | control | Amino acid | Sequence | Genomic |
| Name | IgY | IgY | sequence | length | map |
| Control group (BSA+PBS) | | | | | |
| S1+S2-ECD protein (quality | 7.30 | 6.09 | | | |
| control) | | | | | |
| S2-ECD protein (quality | 7.27 | 10.23 | | | |
| control) | | | | | |
| S1 protein (quality control) | 7.25 | 0.54 | | | |
| RBD protein (quality | 5.68 | 0.33 | | | |
| control) | | | | | |
| NTD (21-35) | 3.78 | <0.05 | RTQLPPAYTNSFTRG | 15 | 840 |
| NTD (141-155) | 3.01 | 0.15 | LGVYYHKNNKSWMES | 15 | 852 |
| NTD (261-275) | 7.41 | <0.05 | GAAAYYVGYLQPRTF | 15 | 864 |
| NTD (291-305) | 7.45 | <0.05 | CALDPLSETKCTLKS | 15 | 867 |
| RBD (411-425) | 3.31 | <0.05 | APGQTGKIADYNYKL | 15 | 879 |
| RBM (461-475) | 7.42 | <0.05 | LKPFERDISTEIYQA | 15 | 884 |
| CTD1 (561-575) | 7.38 | 0.19 | PFQQFGRDIADTTDA | 15 | 894 |
| CTD1 (571-585) | 7.38 | <0.05 | DTTDAVRDPQTLEIL | 15 | 895 |
| CTD2 (581-595) | 7.38 | <0.05 | TLEILDITPCSFGGV | 15 | 896 |
| CTD2 (661-675) | 6.34 | <0.05 | ECDIPIGAGICASYQ | 15 | 904 |
| S1/S2 (741-755) | 3.56 | <0.05 | YICGDSTECSNLLLQ | 15 | 912 |
| S1/S2 (811-825) | 7.43 | 14.16 | KPSKRSFIEDLLFNK | 15 | 919 |
| S1/S2 (821-835) | 3.19 | <0.05 | LLFNKVTLADAGFIK | 15 | 920 |
| HR2 (1,161-1,175) | 7.38 | <0.05 | SPDVDLGDISGINAS | 15 | 954 |
| HR2-TM (1,201-1,215) | 7.19 | <0.05 | QELGKYEQYIKWPWY | 15 | 958 |
| ORF1ab (1,361-1,375) | 3.35 | <0.05 | SNEKQEILGTVSWNL | 15 | 190 |
| OFR1ab (6,411-6,425) | 3.15 | <0.05 | HHANEYRLYLDAYNM | 15 | 695 |
| ORF10 (21-38) | 4.93 | 1.46 | MNSRNYIAQVDVVNF | 18 | 991 |
| | | | NLT | | |
| ORF7a (1-15) | 4.01 | <0.05 | MKIILFLALITLATC | 15 | 992 |
| ORF7a (111-121) | 6.52 | 6.46 | TLCFTLKRKIE | 11 | 1003 |

In the above 20 types of amino acid sequences having an efficient neutralizing effect, 15 types of which were identified to be positioned in the S-ECD region, 5 types of which were positioned in the non-structural protein (NSP) region, and the amino acid sequences were shown in Table 2.

### APPLICATION EXAMPLE

### Application Example 1

Detection of an inhibitory effect of the anti-novel coronavirus neutralizing antibodies N-IgY-pAbs on the novel coronavirus.

Neutralizing activity testing was performed using live novel coronavirus under biosafety level 3 (BSL-3) laboratory conditions.
1) Vero cells (10⁵/mL) were inoculated into a 96-well plate, 100 μl per well, and incubated overnight at 37° C. in a CO₂ incubator. When a cell intensity reached to 80%-90%, washed and set aside.
2) Different concentrations of the purified anti-novel coronavirus neutralizing antibodies N-IgY-pAbs (747 μg/mL; 74.7 μg/mL; 7.47 μg/mL; 0.747 μg/mL) were mixed with 100 TCID50 of live novel coronavirus, respectively. Thus a mixture solution of antibody and virus was obtained, with a total volume of 100 μL, and incubated at 37° C. for 1 hour. Blank wells of DMEM culture medium were set as negative controls.
3) The 96-well plate containing Spare Vero cells was taken, a supernatant of which was discarded, 100 μL of the mixture solution of antibody and virus with each antibody concentrationin step 2) was added into the cells, then 100 uL of maintenance medium (DMEM culture medium containing 2% fetal bovine serum) was added, and incubated at 37° C. for 48 hours.
4) A cell culture supernatant was collected, a total RNA was extracted according to kit instructions (Roche high purity viral RNA purification kit, item number: 11859992001), a copy number of the new coronavirus in the total RNA was detected by using a novel coronavirus nucleic acid testing kit (fluorescent quantitative PCR method, produced by Berger medical science and technology Co., Ltd., Shanghai, China). Primers and probes of the novel coronavirus targeted open reading frame lab (ORF lab) and nucleocapsid protein (N protein), and an inactivation rate of the new coronavirus in a sample was calculated according to a formula provided by a kit manufacturer and was shown in Table 3. A concentration of the N-IgY-pAbs after optimization was 7 μg/mL.

TABLE 3
inactivation rate of the new coronavirus in Neutralization test

| Repeated | | Inactivation | Inactivation |
|---|---|---|---|
| detection of | Purified N-IgY- | rate % | rate % |
| three times | pAbs μg/ml | (ORFlab gene) | (N gene) |
| N-IgY-pAbs | 10 × (75 | 98.45 ± 2.51% | 99.3 ± 0.91% |
| | μg/ml) | | |
| | 100 × (7.5 | 98.80 ± 3.42% | 99.5 ± 0.34% |
| | μg/ml) | | |
| | 1,000 × (0.75 | 86.1 ± 31.71% | 77.99 ± 36.12% |
| | μg/ml) | | |
| Control 1 | Positive: virus + | 14.792 ± 1.52% | 16.12 ± 1.11% |
| (without | DMEM | | |
| antibody) | | | |
| Control 2 | Positive: virus + | 14.1213 ± 1.32% | 15.22 ± 1.41% |
| (without | PBS | | |
| antibody) | | | |

### Application Example 2

A spray formulation of the anti-novel coronavirus neutralizing antibodies N-IgY-pAbs and stability thereof.
1) Preparation of the spray formulation of the anti-novel coronavirus neutralizing antibodies N-IgY-pAbs: Formulation ingredients included the anti-novel coronavirus neutralizing antibodies N-IgY-pAbs; NaCl or mannitol; and Sterile deionized water or water for injection. In particular, a concentration of the anti-novel coronavirus neutralizing antibodies N-IgY-pAbs ranged in 3-15 μg/ml, a mass concentration of NaCl was 0.9% when the NaCl was added; and a concentration of the mannitol ranged in 10-30 g/L when the mannitol was added.

Specifically, a preparation method in this Example was as follows.
step 1: The anti-novel coronavirus neutralizing antibodies N-IgY-pAbs was filtered through a 0.22 μm filter membrane.
step 2: 10 μg of anti-novel coronavirus neutralizing antibodies N-IgY-pAbs solution was taken, the sterile deionized water was added to make a dilution ratio of the anti-novel coronavirus neutralizing antibodies N-IgY-pAbs solution and sterile deionized water 1:3, the NaCl was added to adjust a final concentration to be 0.9%, and pH of which was adjusted to 7.4.
step 3: Filtered by using a 0.22 m vacuum filtration bottle, and then packaged in sterile bottles.
2) Stability evaluation: The spray formulation of the anti-novel coronavirus neutralizing antibodies N-IgY-pAbs was stored at 4° C. for 6-12 months. In this Example, it was stored for 12 months. The stability was evaluated by using enzyme linked immunosorbent assay (ELISA). The specific step was as follows.
a) Antigen coating: Antigens of S protein RBD or S-ECD were diluted to a concentration of 0.5 μg/mL using a coating solution, 100 uL per well, and coated overnight at 4° C.;
b) Sealing after discarding solution and washing plate: 200 μL of a sealing solution was added to each well, and sealed at 37° C. for 1 hour;
c) Antibody reaction after discarding solution and washing plate: A sample was gradient diluted with antibody diluent, 100 μL of antibody to be detected was added to each well, and reacted at 37° C. for 1 hour;
d) The plate was washed three times;
e) Adding biotinylated secondary antibody: 100 μL of biotinylated second antibody diluted 250 times was added to each well, and reacted at 37° C. for 1 hour;
f) The plate was washed three times;
g) 100 μL of SA-HIRP working solution was added to each well, and reacted at 37° C. for 1 hour;
h) The plate was washed three times;
i) Color development: 100 μL of TMB color solution was added to each well, 37° C., for 10 minutes;
j) 70 μL of stop solution was added to stop the reaction; and
k) An absorbance was measured at 450 nm wavelength by using microplate reader.

A stability data of the spray formulation of the anti-novel coronavirus neutralizing antibodies N-IgY-pAbs was shown in Table 4. Results showed that the spray formulation of the anti-novel coronavirus neutralizing antibodies N-IgY-pAbs could be stored at 4° C. for at least 12 months, and there was no obvious change in OD value (p>0.05).

TABLE 4
Stability of the spray formulation of the anti-novel
coronavirus neutralizing antibodies N-IgY-pAbs

| | Spray formulation of anti-novel coronavirus | | | | | |
|---|---|---|---|---|---|---|
| | neutralizing antibodies N-IgY-pAbs | | | | | |
| | RBD, 0.5 μg/ml | | | S-ECD, 0.5 μg/ml | | |
| time | 0 month | 6 months | 12 months | 0 month | 6 months | 12 months |
| mean | 5.27 | 5.64 | 5.41 | 5.43 | 5.59 | 5.58 |
| standard deviation | 0.27 | 0.38 | 0.34 | 0.40 | 0.22 | 0.40 |
| CV | 5.12% | 6.72% | 6.28% | 7.35% | 3.93% | 7.22% |
| P value | / | 0.073 | 0.190 | / | 0.234 | 0.241 |
| blank control | 0.18 | / | 0.16 | 0.17 | / | 0.12 |

### Application Example 3

Neutralizing effect of the spray formulation of the anti-novel coronavirus neutralizing antibodies N-IgY pAbs prepared in Application Example 2 on novel coronavirus variant Omicron virus.

After identification by BSL-3 Laboratory of Shenzhen Third People's Hospital (Report No. SZSY202201), an inhibition rate of the spray formulation of the anti-novel coronavirus neutralizing antibodises N-IgY pAbs (No. SSTK08) prepared in Application Example 2 against novel coronavirus Omicron virus strain was more than 99%. Specifically, an identify method included the following steps. The neutralizing effect of the spray formulation at a 2-fold dilution on Vero E6 cells infected with the novel coronavirus variant Omicron was detected by using a focus reduction neutralization test (FRNT) method. Results showed that, at a concentration of 305 μg/ml, neutralizing activities of three repeated wells were 99.3%, 100%, and 99.3%, respectively.

### Application Example 4

Neutralizing effect of the spray formulation of the anti-novel coronavirus neutralizing antibodies N-IgY pAbs (Sample No. SSKT-WH06) prepared in Application Example 2 on novel coronavirus variant Delta virus.

After identification by P3 Laboratory of State Key Laboratory of Virology of Wuhan University, the inhibition rate of the spray formulation of the anti-novel coronavirus neutralizing antibodies N-IgY pAb (Test sample No. SSKT-WH06) against novel coronavirus Delta virus strain (B.1.617.2) was 99.94%.

Whole proteome chip detection results showed that the anti-novel coronavirus had a property of specifically recognizing a variety of target amino acid sequences, these target amino acid sequences can be cooperated with each other, so as to promoting an interaction between the anti-novel coronavirus neutralizing antibodies N-IgY pAbs and the virus receptor binding domain (RBD), and blocking a binding of the RBD and the host receptor angiotensin converting enzyme 2 (ACE2), thereby effectively neutralizing the novel coronavirus.

The anti-novel coronavirus N-IgY pAbs showed a neutralizing and inhibiting effect on the novel coronavirus. The spray formulation prepared by anti-novel coronavirus N-IgY pAbs could be stored stably at 4° C. for at least 12 months.

The above Application Examples showed that, with a help of the above 20 types of amino acid sequences in the present application, one or more of the above amino acid sequences are used as a core to perform corresponding adjustment or modification. The material used for modification included but was not limited to nanomaterial, fluorescent material, enzyme, biotin, and specific protein. It could be applied to the detection of the novel coronavirus, and the detection included but was not limited to ELISA detection, immunochemiluminescence detection, and immunofluorescence detection. It could also be applied to a design of targets of novel coronavirus vaccines, including a design of vaccine immune antigens and an evaluation of vaccine performance.

With the help of the above 20 types of amino acid sequences in the present application, using one or more of the amino acid sequences as the core, it could be applied to a design of therapeutic targets of the novel coronavirus, including a design of targets of therapeutic antibodies and a design of targets of non-antibody therapeutic drugs.

The specific examples are only provided for an explanation of the present application, not intended to impose any limitation to the present application. Those skilled in the art can make modifications to the embodiment as needed without paying creative contribution after reading this specification, which, as long as falls within the scope of the claims of the present application, shall be protected by a patent law.

What is claimed is:

1. A core amino acid sequence group capable of target recognizing anti-novel coronavirus neutralizing antibodies N-IgY-pAbs, comprising the following amino acid sequences:
SNTD:
²¹RTQLPPAYTNSFTRG³⁵,
¹⁴¹LGVYYHKNNKSWMES¹⁵⁵,
²⁶¹GAAAYYVGYLQPRTF²⁷⁵
and
²⁹¹CALDPLSETKCTLKS³⁰⁵;
S-RBD:
⁴¹¹APGQTGKIADYNYKL⁴²⁵
and
⁴⁶¹LKPFERDISTEIYQA⁴⁷⁵;
S-CTD1:
⁵⁶¹PFQQFGRDIADTTDA⁵⁷⁵,
⁵⁷¹DTTDAVRDPQTLEIL⁵⁸⁵
and
⁵⁸¹TLEILDITPCSFGGV⁵⁹⁵;
S-CTD2:
⁶⁶¹ECDIPIGAGICASYQ⁶⁷⁵;
S-HR2-TM:
¹²⁰¹QELGKYEQYIKWPWY¹²¹⁵
S1/S2 border region:
⁷⁴¹YICGDSTECSNLLLQ⁷⁵⁵
⁸¹¹KPSKRSFIEDLLFNK⁸²⁵
and
⁸²¹LLFNKVTLADAGFIK⁸³⁵;
S-HR2:
¹¹⁶¹SPDVDLGDISGINAS¹¹⁷⁵;
and
S-HR2-TM:
¹²⁰¹QELGKYEQYIKWPWY¹²¹⁵,
positioned in an S-ECD region and numbered according to a protein sequence; and
ORF1ab:
¹³⁶¹SNEKQEILGTVSWNL¹³⁷⁵;
ORF1ab:
⁶⁴¹¹HHANEYRLYLDAYNM⁶⁴²⁵;
ORF10:
²¹MNSRNYIAQVDVVNFNLT³⁸;
and
ORF7a:
¹MKIILFLALITLAT¹⁵
and
¹¹¹TLCFTLKRKTE¹²¹,
positioned in a non-structure protein region and numbered according to a protein sequence.

2. A core amino acid sequence group capable of target recognizing anti-novel coronavirus neutralizing antibodies N-IgY-pAbs, wherein the core amino acid sequence group consists of the following amino acid sequences:
SNTD:
²¹RTQLPPAYTNSFTRG³⁵,
¹⁴¹LGVYYHKNNKSWMES¹⁵⁵,
²⁶¹GAAAYYVGYLQPRTF²⁷⁵
and
291CALDPLSETKCTLKS³⁰⁵;
S-RBD:
⁴¹¹APGQTGKIADYNYKL⁴²⁵
and
⁴⁶¹LKPFERDISTEIYQA⁴⁷⁵;
S-CTD1:
⁵⁶¹PFQQFGRDIADTTDA⁵⁷⁵,
⁵⁷¹DTTDAVRDPQTLEIL⁵⁸⁵
and
⁵⁸¹TLEILDITPCSFGGV⁵⁹⁵;
S-CTD2:
⁶⁶¹ECDIPIGAGICASYQ⁶⁷⁵;
S-HR2-TM:
¹²⁰¹QELGKYEQYIKWPWY¹²¹⁵
S1/S2 border region:
⁷⁴¹YICGDSTECSNLLLQ⁷⁵⁵
⁸¹¹KPSKRSFIEDLLFNK⁸²⁵
and
⁸²¹LLFNKVTLADAGFIK⁸³⁵;
S-HR2:
¹¹⁶¹SPDVDLGDISGINAS¹¹⁷⁵;
and
S-HR2-TM:
¹²⁰¹QELGKYEQYIKWPWY¹²¹⁵,
positioned in an S-ECD region and numbered according to a protein sequence; and
amino acid sequences positioned in a non-structure protein region are numbered according to a protein sequence as follows:
ORF1ab:
¹³⁶¹SNEKQEILGTVSWNL¹³⁷⁵;
ORF1ab:
⁶⁴¹¹HHANEYRLYLDAYNM⁶⁴²⁵;
ORF10:
²¹MNSRNYIAQVDVVNFNLT³⁸;
and
ORF7a:
¹MKIILFLALITLAT¹⁵
and
¹¹¹TLCFTLKRKTE¹²¹,
positioned in a non-structure protein region and numbered according to a protein sequence.

3. The core amino acid sequence group capable of target recognizing anti-novel coronavirus neutralizing antibodies N-IgY-pAbs according to claim 1, wherein the amino acid sequence is an adjusted or modified amino acid sequence, and a material used for modification comprises any one or more selected from a group consisting of nanomaterial, fluorescent material, enzyme, biotin, and a specific protein.

4. Use of the core amino acid sequence group capable of target recognizing anti-novel coronavirus neutralizing antibodies N-IgY-pAbs according to claim 1 in a detection of novel coronavirus.

5. The use of the core amino acid sequence group capable of target recognizing anti-novel coronavirus neutralizing antibodies N-IgY-pAbs according to claim 4, wherein the detection comprises enzyme-linked immunosorbent assay (ELISA) detection, chemiluminescence immunoassay detection and immunofluorescence method detection.

6. Use of the core amino acid sequence group capable of target recognizing anti-novel coronavirus neutralizing antibodies N-IgY-pAbs according to claim 1 in designing a therapeutic target for novel coronavirus.

7. The use of the core amino acid sequence group capable of target recognizing anti-novel coronavirus neutralizing antibodies N-IgY-pAbs according to claim 6, wherein the therapeutic target comprises a target of therapeutic antibody and a target of non-antibody therapeutic drug.
